(19) United States
(12) Patent Application Publication
(10) Pub. No.: US 2023/0175944 A1
(43) Pub. Date: Jun. 8, 2023

(54) METHOD FOR DETERMINING AT LEAST ONE CHARACTERISTIC VARIABLE OF A PARTICLE SIZE DISTRIBUTION AND A DEVICE COMPRISING A MEASURING APPARATUS

(71) Applicant: TEWS Elektronik GmbH & Co. KG, Hamburg (DE)

(72) Inventors: Udo Schlemm, Hamburg (DE); Hendrik Richter, Hamburg (DE)

(73) Assignee: TEWS Elektronik GmbH & Co. KG, Hamburg (DE)

(21) Appl. No.: 17/928,665

(22) PCT Filed: Sep. 4, 2020
(86) PCT No.: PCT/EP2020/074756

§ 371 Date: Nov. 30, 2022

(30) Foreign Application Priority Data
Jun. 3, 2020 (DE) 10 2020 114 726.0

(51) Int. Cl. G01N 15/02

(52) U.S. Cl. CPC G01N 15/0205

(57) ABSTRACT

A method for determining at least one characteristic variable of a particle size distribution in a moving flow of particles includes structuring at least one microwave resonator to determine at least two measured values for the moving flow of particles. At least one quantile of the particle size distribution is further determined from the at least two measured values. Moreover, a device for generating a moving flow of particles includes a measuring apparatus having at least one microwave resonator that determines at least two measured values for the flow of particles. The measuring apparatus evaluates at least one quantile of a particle size distribution from the at least two measured values of the microwave resonator.

### CROSS REFERENCE TO RELATED INVENTION

This application is a national stage application pursuant to 35 U.S.C. § 371 of International Application No. PCT/EP2020/074756, filed on Sep. 4, 2020, which claims priority to, and benefit of, German Patent Application No. 10 2020 114 726.0, filed Jun. 3, 2020, the entire contents of which are hereby incorporated by reference.

### TECHNOLOGICAL FIELD

The present disclosure is directed to a method for determining at least one characteristic variable of a particle size distribution in a moving flow of particles. The disclosure is further directed to a device for generating a moving flow of particles comprising a measuring apparatus for determining at least one characteristic variable of a particle size distribution in the moving flow of particles.

### BACKGROUND

WO 2009/030314 discloses a method for measuring a moisture value of dielectric substances using at least one resonator. In this method, a shift of the resonance frequency is evaluated for each of at least two resonance modes that have different resonance frequencies from one another and a density-independent moisture value is calculated from the measured shift of the resonance frequency. The particular advantage of this method is intended to be that a damping value is no longer required as a measure of the moisture content during determination of the moisture value. Instead, a density-independent moisture value is calculated with a high degree of reliability from at least two shifts of the resonance frequency that occur at different resonance frequencies. By drawing on the frequency shift at two resonance frequencies, a particle size D can be calculated without the need for damping characteristic values. In practice, it has been shown that a particle size D in a moving flow of particles can only be determined in a very unreliable manner.

DE 101 11 833 C1 discloses a measuring probe for the in-line determination of the size of moving particles in transparent media. The measuring probe comprises a tubular measuring probe body, into which individual particles of the moving particles enter and are optically measured. For this purpose, the particles are separated using a dispersing medium.

DE 3 241 544 A1 discloses a method for monitoring and/or control during drying, granulation, instantiation, pan-coating, and film-coating processes. In the known method, the moisture content of the exhaust air and the moisture content of the supply air is measured and the resulting moisture difference is used to control the work process.

### BRIEF SUMMARY OF THE INVENTION

The object of the invention is to reliably measure at least one characteristic variable of a particle size distribution in a moving flow of particles. Furthermore, the object of the invention is to provide a measuring apparatus of this kind for a device for generating a moving flow of particles.

The method according to the invention is intended for determining at least one characteristic variable of a particle size distribution in a moving flow of particles. At least one microwave resonator which provides at least two measured values for the flow of particles in each case is used for the determination. An important feature is that a fineness characteristic and/or a quantile of the particle size distribution is determined by means of the characteristic variable. Unlike in the prior art, in which, for example, a mean particle size or maybe another mean variable was determined, according to the invention a proportion of a distribution is determined using the fineness characteristic, i.e., for example, the number, the mass, or another variable is evaluated with regard to how large the proportion of particles is that are smaller than or equal to the fineness characteristic. Therefore, a quantitative value itself is not taken into account, but rather a proportion up to the quantitative value. Moreover, the quantile defines a threshold value at which a certain proportion of the values is smaller than the quantile, while the rest is larger. For example, the 25% quantile denotes the value for which 25% of all values are smaller than this value and 75% thereof are larger. According to the invention, a special step for evaluating particle size distributions using a microwave resonator consists in not looking at specific values or average values, but rather comprises evaluating the measured variables in such a way that the contributions of all moving particles that are smaller than the fineness characteristic or rather the quantile to be examined are always considered, too. In addition to the use of multiple microwave resonators, it is also possible to use microwave resonators which comprise two or more resonance modes. Two measured values for the flow of particles can then be provided for each resonance mode.

In a preferred embodiment, it has proven advantageous to take a resonance frequency shift and a broadening of the resonance curve into account as the two measured values of the microwave resonator. The resonance frequency shift and the broadening of the resonance curve (B) as the measured value are, in principle, density-dependent variables, whereas the quotient of the two measured values provides a variable that is independent of the mass or rather density. In particular, by taking into account the fineness characteristic or rather a quantile, the use of the two measured values of the microwave resonator with the resonance frequency shift and broadening of the resonance curve is particularly advantageous. Other measured values of the microwave resonator that provide information about the damping of the resonance may also be used instead of the broadening of the resonance curve.

In an embodiment of the method, at least one temperature of the flow of particles is evaluated. The temperature of the flow of particles results from the temperature of the supplied air and the evaporation heat. The temperature of a moist flow of particles is therefore lower than that of a dry flow of particles (at a constant fill level and amount of air supplied) on account of the more pronounced evaporation at a constant supply air temperature.

Preferably, for a moving flow of particles in a fluidized bed, at least one of the following variables is evaluated as further measured variables: the amount of air supplied and the fill level of the fluidized bed. The amount of air supplied is set on fluidized bed dryers depending on the relevant process and is usually also varied during the processes. It can be expressed, for example, in cubic meters/hour [m\hat{ }3/h]. The fill level in a fluidized bed system indicates, for example, how many kilograms [kg] of material there are in the fluidized bed system.

Preferably, it has been shown that the at least one fineness characteristic and/or one quantile can be determined very accurately by means of linear approximation of the evaluated measured variables. This means that the measured variables used are additionally incorporated into the determination of the fineness characteristic and/or quantile as a simple linear combination with a constant term. Said linear approximation also makes it clear that, for the microwave measured values, the determination of the fineness characteristic or quantile are the suitable characteristic variables of the particle size distribution. Of course, further variables such as the mean particle weight or mean particle diameter can also be determined from these variables. What is crucial, however, is that primarily the fineness characteristic and/or quantile are ascertained.

Preferably, it is possible to consider different particle size distributions. On the one hand, it is possible to take into account a number distribution sum, a length distribution sum, an area distribution sum, or a volume/mass distribution sum. For a good understanding of the particle size distribution, it is particularly relevant to determine multiple variables at the same time. For example, a number distribution sum and a volume distribution sum could be taken into consideration with the same measured values but different coefficients in the linear combination. It is also possible to determine multiple quantiles and/or fineness characteristics of a distribution sum.

The object according to the invention is also achieved by a device for generating a moving flow of particles having the features of claim 10. The device comprises a measuring apparatus for determining at least one characteristic variable of a particle size distribution in the moving flow of particles. The measuring apparatus comprises at least one microwave resonator which provides at least two measured values for the flow of particles in each case. The at least two measured values are preferably the resonance frequency shift and a broadening of the resonance curve. Furthermore, the measuring apparatus is configured to evaluate at least one fineness characteristic and/or one quantile of the particle size distribution from the two measured values of the microwave resonator. The measuring apparatus, to which the measured values of the at least one microwave resonator are applied, may be arranged spatially together with the microwave resonator or separately therefrom. The microwave resonator may be provided for generating two or more resonance modes, wherein the at least two measured values are recorded in each of the resonance modes.

Moreover, the measuring apparatus is preferably configured to additionally evaluate a temperature of the flow of particles.

Preferably, the measuring apparatus is arranged such that the measured variables are measured in a fluidized bed. In particular, when used in a fluidized bed, the measuring apparatus is configured to evaluate at least one of the following variables, such as the amount of air supplied and the fill level of the fluidized bed. The amount of air supplied and the fill level have a significant influence on the microwave measurement and are therefore preferably taken into consideration for the determination of the fineness characteristic and/or the quantile to be determined for a flow of particles.

The measuring apparatus is preferably oriented in such a way that the fineness characteristic and/or the quantile are determined for a number distribution sum, a length distribution sum, an area distribution sum, or a volume distribution sum and/or a mass distribution sum. The important aspect here is that the measuring apparatus can also determine multiple fineness characteristics and quantiles at the same time.

Therefore, if multiple fineness characteristics and/or quantiles are recorded over time, the processes taking place in the moving layer can be determined in a reliable manner.

### BRIEF DESCRIPTION OF THE DRAWINGS

The above invention will be explained in more detail below with the aid of some measured values. In the drawings:

FIG. 1 graphically illustrates an embodiment of the temporal development of three fineness characteristics relating to the number distribution sum;

FIG. 2 graphically illustrates an embodiment of three fineness characteristics relating to the volume distribution sum; and

FIG. 3 graphically illustrates an embodiment of a temporal development for a mean diameter of the particles.

### DETAILED DESCRIPTION OF THE INVENTION

Fluidized bed processes are used in many different technical fields. One important field of application is the pharmaceutical production process, in the manufacture of discrete active ingredient units that can, for example, be pressed into tablets or filled into capsules. In this case, a granulation process with a subsequent fluidized bed drying process is used. During the granulation process, the present pharmaceutical powder mixture is processed into granules with a defined particle size while an, often aqueous, solution is sprayed in. In the subsequent fluidized bed drying process, the granules are dried to a defined target moisture content. Both processes can take place in separate systems, but it is also possible to perform them in a combined manner in one system. In addition to the moisture content, an important parameter for characterizing the quality of the substrate produced is the mean particle size of the granules. In addition to process and end product monitoring, measuring the particle size distribution also makes it possible to identify operational malfunctions, for example at the spray nozzles. It is important to bear in mind here that not only is a mean particle diameter crucial, but rather knowledge of the overall particle size distribution is helpful for assessing the process. If, for example, large particles are present, i.e. so-called “oversize”, this does not necessarily lead an associated increase in the average particle diameter, but it is nevertheless prejudicial to further processing processes. Likewise, a high fine content can occur, for example due to mechanical stress with insufficient stability of the granules, and also poses difficulties for subsequent further processing. The properties of the granules with their particle size distribution have a direct influence on the subsequent processing and also on the properties, for example, of the finished tablet with regard to the dissolution kinetics thereof as well as uniform release of the active ingredient content.

Currently, the measurement of the particle size directly during the fluidized bed process is mainly done using a laser method, as described, for example, in DE 10 111 833 C1. The disadvantage of the optical method is that it is extremely sensitive to contamination and, additionally, is only suitable for optically measuring the particle size and not for simultaneously measuring the moisture content.

When measuring in a flow of particles, a distinction can be made between the particle (disperse phase) and its surrounding medium (continuous phase). In the fluidized bed, the drying granules constitute the particles, whereas the surrounding air constitutes the continuous medium. It is typical to decide grains, drops, or bubbles based on an equivalent diameter to be measured and to classify them into selected classes according to their size. In order to represent a particle size distribution, the proportions of the respective particle classes in the disperse phase are determined.

Different types of quantity are known. If the particles are counted, then the quantity is the number. However, if they are weighed, it is the mass or rather, in the case of a homogeneous density, the volume. Other types of quantity are derived from length, projection area, and surface area. In general, the following can be distinguished:

| Type of quantity | Index R |
|---|---|
| Number | 0 |
| Length | 1 |
| Area | 2 |
| Volume (mass) | 3 |

It is typical to use a standardized quantitative measure for graphical representation, such that the dependency of the proportions on the total quantity used are eliminated. When using the above-mentioned indices, a number distribution sum Q0 and, for example, a volume distribution sum Q3 are obtained. If X denotes a particle size as an equivalent diameter, in the usual notation this produces, for example, X10,0 for the fineness characteristic at which the distribution sum Q0 assumes the value 10%. In other words, the 10% quantile of the distribution function is at the value X10,0, which means that 10% of all particles have this or a lesser diameter.

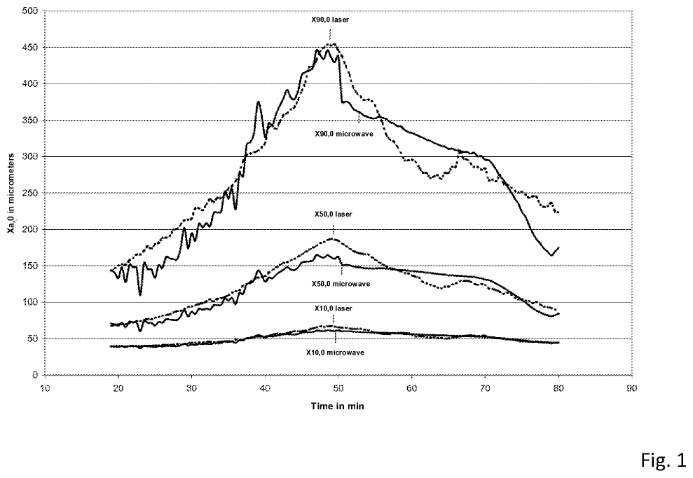

In FIG. 1, the measurement results of the method according to the invention are plotted by means of the solid line. The upper curve X90,0 is shown for a process duration of 20-80 minutes. The y-axis shows the diameter of the particles. A value of approximately 400 μm in the curve X90,0, as occurs, for example, shortly before 50 minutes and shortly after 50 minutes of process duration means that 90% of the particles have a diameter of less than or equal to 400 μm. The curve X50,0 indicates the equivalent diameter that 50% relative to the number have, for example a diameter of less than 150 μm. The fineness characteristic X10,0 indicates the maximum diameter of the 10% smallest particles. The temporal development of these three fineness characteristics gives a good indication of the grain size distribution. If, for example, the value for X10,0 is too small, it can be deduced that 10% of the particles relative to the number are smaller than this value and are thus possibly too small. Equally, an excessively large value for X90,0 can be an indication that isolated large grains are oversized.

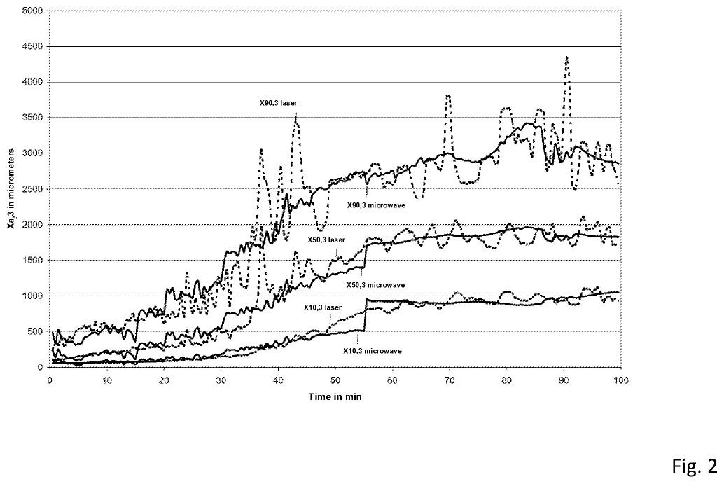

FIG. 2 shows the fineness characteristic in μm in relation to the volume distribution sum. The curve X_{90,3} indicates the quantiles for the volume distribution sum. This means that X_{90,3} denotes, for example, the largest diameter of the particles, which make up 90% of the total volume.

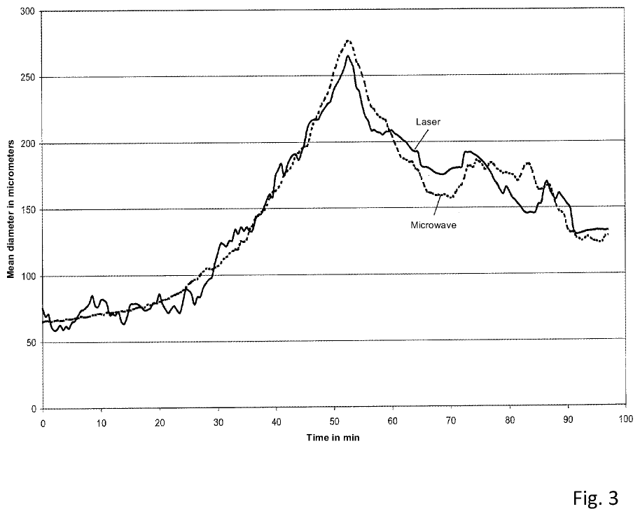

FIG. 3 shows how the mean diameter of the particles develops over time. The mean particle diameter increases constantly during granulation and decreases during the drying phase due to the constant collision of the particles. The transition between granulation and the drying phase takes place at approximately 52 to 55 min.

FIG. 1-3 each show parallel optical measurements, which are referred to as laser measurement. A comparison shows that values can also be reliably obtained using a microwave resonator here.

The following approaches have proven successful for the evaluation of the measured values:

Xa,0=a₁·A+a₂·B+a₃·L+a₄·T+a₅·F+a₀

Xa,3=b₁·A+b₂·B+b₃·L+b₄·T+b₅·F+b₀

wherein, here, X_{a,0} denotes the fineness characteristics to the quantiles a of the number distribution sum in μm and X_{a,3} denotes the fineness characteristics to the quantiles a of the volume distribution sum in μm and aᵢ and bᵢ each denote the calibration coefficients. The measured variables to be evaluated are A for the resonance frequency shift of a resonance mode in MHz, B a broadening of the resonance curve of the same resonance mode in MHz, L the amount of air supplied to the fluidized bed in m³/h, T the product temperature in degrees Celsius, and F the fill level of the fluidized bed system in kg.

1-17. (canceled)

18. A method for determining at least one characteristic variable of a particle size distribution in a moving flow of particles, comprising:
structuring at least one microwave resonator to determine at least two measured values for the moving flow of particles; and
determining at least one quantile of the particle size distribution from the at least two measured values.

19. The method according to claim 18, wherein the at least two measured values of the microwave resonator correspond to a resonance frequency shift (A) and a broadening of a resonance curve (B).

20. The method according to claim 18, further comprising evaluating at least one temperature of the moving flow of particles.

21. The method according to claim 18, wherein the moving flow of particles is present in a fluidized bed.

22. The method according to claim 21, further comprising evaluating at least one of: (i) an amount of air supplied to the fluidized bed; and (ii) a fill level of the fluidized bed.

23. The method according to claim 18, further comprising determining at least one of: (i) a fineness characteristic; and (ii) a quantile, is linearly approximated using the at least two measured variables.

24. The method according to claim 23, wherein the quantile relates to one of a number distribution sum, a length distribution sum, an area distribution sum, a volume distribution sum, or a mass distribution sum.

25. The method according to claim 23, wherein multiple quantiles are recorded over time.

26. A device for generating a moving flow of particles, comprising:
a measuring apparatus comprising at least one microwave resonator configured to determine at least two measured values for the flow of particles,
wherein the measuring apparatus is configured to evaluate at least one quantile of a particle size distribution from the at least two measured values of the microwave resonator.

27. The device according to claim 26, wherein the measuring apparatus is further configured to measure a temperature of the flow of particles.

28. The device according to claim 26, wherein the at least two measured values are measured in a fluidized bed.

29. The device according to claim 28, wherein the measuring apparatus is configured to evaluate at least one of: (i) an amount of air supplied to the fluidized bed; and (ii) a fill level of the fluidized bed.

30. The device according to claim 29, wherein the measuring apparatus is configured to linearly approximate the quantile using: (i) the amount of air supplied to the fluidized bed; and (ii) the fill level of the fluidized bed.

31. The method according to claim 26, wherein the measuring apparatus is configured to determine the quantile for one of: a number distribution sum, a length distribution sum, an area distribution sum, a volume distribution sum, or a mass distribution sum.

32. The method according to claim 26, wherein the measuring apparatus is configured to record multiple quantiles over time.
